(19) United States
(12) Patent Application Publication
(10) Pub. No.: US 2020/0129928 A1
(43) Pub. Date: Apr. 30, 2020

(54) WATER TREATMENT CHEMICAL FOR MEMBRANE, AND MEMBRANE TREATMENT METHOD

(71) Applicant: KURITA WATER INDUSTRIES LTD., Tokyo (JP)

(72) Inventors: Kazuki ISHII, Tokyo (JP); Nozomu IKUNO, Tokyo (JP)

(21) Appl. No.: 16/493,588

(22) PCT Filed: Mar. 2, 2018
(86) PCT No.: PCT/JP2018/007995

§ 371 Date: Sep. 12, 2019

(30) Foreign Application Priority Data
Mar. 15, 2017 (JP) 2017-050167

(51) Int. Cl. B01D 65/08; C02F 1/44; B01D 61/02; C07C 229/16; C07F 9/38

(52) U.S. Cl. CPC B01D 65/08; C02F 1/441; C07F 9/3808; C07C 229/16; B01D 61/02

(57) ABSTRACT

A water treatment chemical for a membrane, being is a water treatment chemical for preventing membrane contamination caused by an organic compound having a phenolic hydroxy group and including an organic amine having two or more nitrogen atoms and having five or more carboxyl groups or four or more phosphate groups. A membrane treatment method including adding the water treatment chemical for a membrane to water to be treated which contains an organic compound having a phenolic hydroxy group and then subjecting the water to be treated to membrane separation treatment.

### TECHNICAL FIELD

The present invention relates to a water treatment chemical for a membrane, which prevents membrane contamination caused by an organic compound having a phenolic hydroxy group in water to be treated when the water to be treated that contains the organic compound having a phenolic hydroxy group is subjected to membrane separation treatment using a reverse osmosis membrane (RO membrane) or the like, and relates to a membrane treatment method using this water treatment chemical for a membrane.

### BACKGROUND ART

Water-saving measures due to enhancement in water recovery ratio by using an RO membrane system in the desalination of seawater or brine or the wastewater recovery system have been positively taken. When the RO membrane system is operated in a high recovery ratio, the components in the RO membrane feed-water are highly concentrated on the RO membrane surface, and therefore, scaling or RO membrane clogging caused by an organic compound has become a problem.

Polyphenol that is an organic compound having a phenolic hydroxy group exists as a humic substance contained in the soil, and is used also as a raw material of food or beverages in food or beverage manufacturing factories, etc.

In surface water and groundwater containing humic substances, wastewater from food/beverage manufacturing factories, etc., an organic compound having a phenolic hydroxy group, such as polyphenol, is contained, and therefore, when such water is subjected to RO membrane treatment as the water to be treated, RO membrane clogging caused by the organic compound having a phenolic hydroxy group in the RO membrane feed-water becomes a problem.

Also when water to be treated that contains polyphenol derived from food/beverage raw materials is subjected to treatment using separation membranes other than the RO membrane, such as a microfiltration membrane, the polyphenol is captured by the filtration membrane, so that there is a possibility that a problem of membrane clogging occurs.

The organic compound having a phenolic hydroxy group, such as a humic substance contained in the soil or polyphenol used as a raw material of food or beverages, causes membrane contamination (fouling) of separation membranes, such as microfiltration membrane (MF membrane), ultrafiltration membrane (UF membrane), nanofiltration membrane (NF membrane) and reverse osmosis membrane (RO membrane). The fouling is a phenomenon that separation object substances and the like present in the membrane feed-water adhere to a membrane surface or inside membrane pores and are deposited thereon. The fouling includes deposition of suspended particles on the membrane surface, layer formation due to adsorption of the particles on the membrane, gelation of a soluble high-molecular substance on the membrane surface, adsorption, deposition or clogging inside the membrane pores, blocking (clogging) of pores by air bubbles, flow path blocking in the module, etc.

The organic compound having a phenolic hydroxy group can be rejected by carrying out coagulation/adsorption treatment in the prior stage of the membrane separation treatment, but in the coagulation treatment, the effect of rejecting an organic compound having a relatively low molecular weight, such as fulvic acid, is low, and in the adsorption treatment, replacement of an adsorbent needs to be periodically carried out.

On this account, instead of such pretreatment, a water treatment chemical that is added to membrane feed-water and prevents membrane contamination caused by an organic compound having a phenolic hydroxy group, such as polyphenol, is desired.

In Patent Literature 1, there has been proposed a method for treating water in which in the prior stage of a membrane separation treatment step, a coagulation treatment step of adding a coagulant composed of an alkali solution of a phenolic resin having a melting point of 130 to 220° C. to water to be treated is carried out. There is a possibility that the phenolic resin that is an organic compound having a phenolic hydroxy group remains and is contained also in coagulation treated water obtained after the coagulation treatment using the coagulant described in Patent Literature 1. Consequently, when this coagulation treated water is subjected to membrane separation treatment, membrane contamination due to the organic compound having a phenolic hydroxy group may occur.

As water treatment chemicals for membranes, a large number of chemicals to cope with scaling caused by scale components such as calcium carbonate and calcium sulfate in the water to be treated have been proposed, but water treatment chemicals to cope with membrane contamination caused by an organic compound having a phenolic hydroxy group have hardly been proposed.

In Patent literature 2, there has been proposed a dispersant for an organic compound having a phenolic hydroxy group, the dispersant containing, as an active substance, a high-molecular compound having a carbonyl group and having a structure in which carbonyl carbon and a nitrogen atom are bonded, such as poly(vinylpyrrolidone) or polyacrylamide.

In order to increase a degree of freedom for selection of chemicals or diversity for practical realization, further development of novel chemicals has been desired.

PTL1: JP 2011-56496 A

PTL2: JP 5867532 B

### SUMMARY OF INVENTION

It is an object of the present invention to provide a water treatment chemical for a membrane, which can effectively prevent membrane contamination caused by an organic compound having a phenolic hydroxy group in water to be treated when the water to be treated that contains the organic compound having a phenolic hydroxy group is subjected to membrane separation treatment using an RO membrane or the like, and a membrane treatment method using this water treatment chemical for a membrane.

The present inventor has found that an organic amine having two or more nitrogen atoms and having five or more carboxyl groups or four or more phosphate groups can effectively disperse an organic compound having a phenolic hydroxy group contained in water to be treated to thereby effectively prevent membrane contamination caused by the organic compound having a phenolic hydroxy group.

The gist of the present invention is as follows.

[1] A water treatment chemical for a membrane, being a water treatment chemical for preventing membrane contamination caused by an organic compound having a phenolic hydroxy group, and comprising an organic amine having two or more nitrogen atoms and having five or more carboxyl groups or four or more phosphate groups.

[2] The water treatment chemical for a membrane according to [1], wherein the organic amine is an organic amine represented by any one of the following (1) to (3):

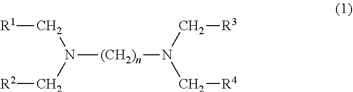

wherein R¹, R², R³ and R⁴ each represent a phosphate group, and n is an integer of 1 or more,

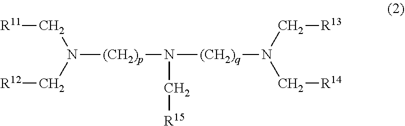

wherein R¹¹, R¹², R¹³, R₁₄, and R¹⁵ each represent a carboxyl group or a phosphate group, and p and q are each independently an integer of 1 or more,

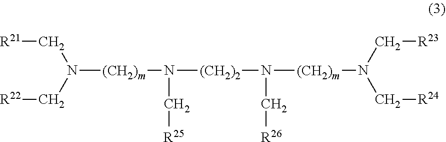

wherein R²¹, R²², R²³, R²⁴, R25 and R²⁶ each represent a carboxyl group, and m is an integer of 1 or more.

[3] The water treatment chemical for a membrane according to [2], wherein n in the general formula (1), p and q in the general formula (2), and m in the general formula (3) are each an integer of 1 to 5.

[4] The water treatment chemical for a membrane according to any one of [1] to [3], wherein a molecular weight of the organic amine is 100 to 1000.

[5] The water treatment chemical for a membrane according to any one of [1] to [4], being a water treatment chemical for a reverse osmosis membrane for carrying out reverse osmosis membrane treatment of water to be treated, the water containing an organic compound having a phenolic hydroxy group, wherein a concentration of non-purgeable organic carbon in concentrated water obtained by the reverse osmosis membrane treatment is 0.01 to 100 mg/L.

[6] The water treatment chemical for a membrane according to [5], wherein a concentration ratio of the reverse osmosis membrane is 3 times or more.

[7] A membrane treatment method comprising adding the water treatment chemical for a membrane according to any one of [1] to [6] to water to be treated, the water containing an organic compound having a phenolic hydroxy group, and then subjecting the water to be treated to membrane separation treatment.

[8] The membrane treatment method according to [7], wherein the water treatment chemical for a membrane is added to the water to be treated in such a manner that the concentration of the organic amine becomes 0.01 to 20 mg/L.

### Advantageous Effects of Invention

According to the present invention, when water to be treated that contains an organic compound having a phenolic hydroxy group is subjected to membrane separation treatment using an RO membrane or the like, the organic compound having a phenolic hydroxy group in the water to be treated is effectively dispersed to prevent membrane contamination caused by the organic compound having a phenolic hydroxy group and to further prevent membrane clogging, whereby a decrease in the amount of water having permeated the membrane is suppressed, and stable and efficient membrane separation treatment can be carried out over a long period of time.

### BRIEF DESCRIPTION OF DRAWING

FIG. 1 is a graph showing results of Examples 1 to 4, Comparative Examples 1 to 6, and Reference Example 1.

### DESCRIPTION OF EMBODIMENT

An embodiment of the present invention is described in detail hereinafter.

The present invention is described below giving, as an example, a case where the water treatment chemical for a membrane of the present invention is applied to membrane separation treatment mainly using an RD membrane, but the water treatment chemical for a membrane of the present invention is also effectively applied to water to be treated (feed-water) in treatment using separation membranes other than the RO membrane, such as MF membrane, UF membrane and NF membrane.

The water treatment chemical for a membrane of the present invention is a water treatment chemical for preventing membrane contamination caused by an organic compound having a phenolic hydroxy group, and contains, as an active substance, an organic amine having two or more nitrogen atoms and having five or more carboxyl groups (—COOH) or four or more phosphate groups (—P(O)(OH)₂).

Details of mechanism that membrane contamination caused by an organic compound having a phenolic hydroxy group can be prevented by the organic amine having two or more nitrogen atoms and having five or more carboxyl groups or four or more phosphate groups, which is used in the present invention, are not clear, but are thought to be as follows.

The carboxyl group or the phosphate group of the organic amine and the phenolic hydroxy group of the organic compound having a phenolic hydroxy group in the water to be treated interact with each other through a hydrogen bond to disperse the organic compound having a phenolic hydroxy group, thereby preventing adhesion of the organic compound to the membrane surface. In that case, if the organic amine is an organic amine having five or more carboxyl groups or four or more phosphate groups, the interaction between this organic amine and the organic compound having a phenolic hydroxy group is large, and a good dispersion effect can be obtained. The upper limit of the number of the carboxyl groups or the phosphate groups in the organic amine is not particularly limited, but in usual, the number of the carboxyl groups is 15 or less, and the number of the phosphate groups is 10 or less.

The organic amine as an active substance of the water treatment chemical for a membrane of the present invention is, for example, an organic amine represented by any one of the following general formulae (1) to (3).

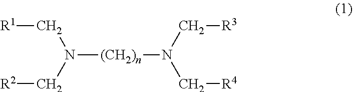

wherein R¹, R², R³ and R⁴ each represent a phosphate group, and n is an integer of 1 or more.

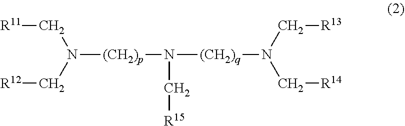

wherein R¹¹, R¹², R¹³, R¹⁴ and R¹⁵ each represent a carboxyl group or a phosphate group, and p and q are each independently an integer of 1 or more.

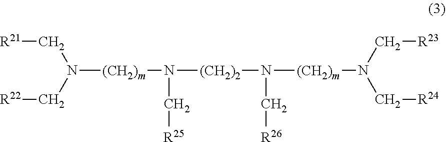

wherein R²¹, R²², R²³, R²⁴, R²⁵ and R²⁶ each represent a carboxyl group, and m is an integer of 1 or more.

n in the general formula (1), p and q in the general formula (2), and m in the general formula (3) are each preferably an integer of 1 to 5, and more preferably 1 to 3.

The molecular weight of the organic amine for use in the present invention is preferably 100 to 1000, and particularly 100 to 800. In the present invention, the molecular weight of the organic amine is a total of atomic weights in its molecular formula.

When the numbers n, p, q and m of the repeating units of the methylene groups in the general formulae (1) to (3) are each in the above range and the molecular weight is in the above range, the organic amine is easily bonded to the organic compound having a phenolic hydroxy group, and the effect of dispersing the organic compound having a phenolic hydroxy group becomes excellent.

Specific examples of the organic amines for use in the present invention include organic amines used in Examples described later, but the organic amines are in no way limited to those described later. The organic amines may be used singly, or may be used in combination of two or more. The organic amine may be used in the form of a salt, such as a sodium salt.

The water treatment chemical for a membrane of the present invention only needs to be one containing the above-described organic amine, and may be one containing other scale preventive agents or slime control agents in addition to the organic amine.

Examples of the organic compounds having a phenolic hydroxy group that are contained in membrane feed-water to which the water treatment chemical of the present invention is applied include the following ones.

The “phenolic hydroxy group” refers to a hydroxy group bonded to an aromatic ring, and examples of the organic compounds having this group include humic acid, fulvic acid, ellagic acid, phenolic acid, tannin, catechin, rutin, anthocyanin, and a synthesized phenolic resin.

The molecular weight (in the case of low-molecular compound) or the weight-average molecular weight (in the case of high-molecular compound) of the organic compound having a phenolic hydroxy group is usually 500 to 1000000, preferably 1000 to 500000, and more preferably 1000 to 100000. When the molecular weight or the weight-average molecular weight of the organic compound having a phenolic hydroxy group is about 500 to 1000000 (preferably 1000 to 100000), it becomes possible to effectively disperse the organic compound by the water treatment chemical for a membrane of the present invention.

When the organic compound having a phenolic hydroxy group is a high-molecular compound such as polyphenol, its weight-average molecular weight is a value in terms of pullulan that is measured by GPC method and calculated using a calibration curve for pullulan standard.

Examples of the water to be treated that contains the organic compound having a phenolic hydroxy group include surface water and groundwater that contain humic substances including polyphenol, and wastewater that is from food/beverage manufacturing factories and contains polyphenol derived from raw materials.

The coagulation treated water described in the aforesaid Patent Literature 1, which is obtained after coagulation treatment using an alkali solution of a phenolic resin as a coagulant and contains a residual phenolic resin, is also preferred water to be treated.

The concentration of the organic compound having a phenolic hydroxy group contained in such water to be treated as above varies depending on the type of the water to be treated, but is usually about 0.01 to 10 mg/L.

pH of the water to be treated that is subjected to membrane treatment is not particularly limited, but is preferably 3.5 to 8.5, more preferably 4.0 to 7.5, and still more preferably 5.0 to 7.0. Therefore, it is preferable to add an acid agent and/or an alkali agent to the water to be treated to thereby adjust its pH to this range, when needed.

The water treatment chemical for a membrane of the present invention can be preferably used as, for example, a water treatment chemical for RO membranes, and by adding this chemical to RO membrane feed-water containing an organic compound having a phenolic hydroxy group especially in such RO membrane separation treatment that the concentration of non-purgeable organic carbon (NPOC) in the resulting concentrated water becomes 0.01 to 100 mg/L, particularly 0.1 to 50 mg/L, or in such RO membrane separation treatment that the concentration ratio is 3 times or more, for example, 3 to 5 times, a good effect can be obtained.

When the water treatment chemical for a membrane of the present invention is added to RO membrane or another membrane feed-water in order to prevent membrane contamination caused by an organic compound having a phenolic hydroxy group, the addition amount of the chemical is preferably such an amount that the concentration of the aforesaid organic amine having two or more nitrogen atoms and having five or more carboxyl groups or four or more phosphate groups becomes 0.01 to 20 mg/L, particularly 0.01 to 10 mg/L. If the addition amount of the organic amine is too small, the effect of dispersing an organic compound having a phenolic hydroxy group due to the organic amine cannot be sufficiently obtained, and if the amount thereof is too large, there is a possibility that the organic amine itself causes membrane contamination.

### EXAMPLES

The effect of the present invention is described in more detail with reference to specific examples. The present invention is in no way limited to the following examples.

Evaluation chemicals which were used in the following Examples and Comparative Examples are set forth in Table 1 described below.

TABLE 1

| | | Molecular |
|---|---|---|
| | Compound name | weight |
| Ex. 1 | ethylenediaminetetramethylenephosphonic | 380 |
| | acid | |
| Ex. 2 | diethylenetriaminepentamethylenephosphonic | 573 |
| | acid | |
| Ex. 3 | diethylenetriaminepentaacetic acid | 436 |
| Ex. 4 | triethylenetetraminehexaacetic acid | 626 |
| Comp. | addition of no chemical | — |
| Ex. 1 | | |
| Comp. | ethylenediaminetetraacetic acid | 292 |
| Ex. 2 | | |
| Comp. | hydroxyiminodiacetic acid | 221 |
| Ex. 3 | | |
| Comp. | amino trimethylene phosphonic acid | 611 |
| Ex. 4 | | |
| Comp. | hydroxyethanephosphonic acid | 206 |
| Ex. 5 | | |
| Comp. | hexamethylenediamine | 492 |
| Ex. 6 | tetra(methylenephosphonic acid) | |

Test methods in Examples and Comparative Examples are as follows.

### <Preparation of Test Solution>

An aqueous solution containing a Canadian Fulvic Acid solution (adjusted so that the UV260 value became 0.5) as an organic compound having a phenolic hydroxy group, calcium chloride (100 mg/L), 30 mg/L of Ca and 2 mg/L of the evaluation chemical was prepared, and further, pH of the aqueous solution was adjusted to 6.5 to 7.0 by using a small amount of a sodium hydroxide aqueous solution or a sulfuric acid aqueous solution, thereby preparing a test solution I.

The Canadian Fulvic Acid was an organic compound having a phenolic hydroxy group and having a molecular weight of 10000, and the concentration of the organic compound having a phenolic hydroxy group in the test solution I was 7.4 to 7.6 mg/L.

Separately, a test solution II was prepared in the same manner as for the test solution I, except that the Canadian Fulvic Acid and the evaluation chemical were not added (Reference Example 1).

Moreover, a test solution III was prepared in the same manner as for the test solution I, except that the evaluation chemical was not added (Comparative Example 1).

### <RO Membrane Pass Test>

Using the above test solutions I to III, RO membrane pass tests were carried out under the following conditions.

RO membrane: polyamide-based RO membrane (“ES20” manufactured by Nitto Denko Corporation)

Temperature: 24 to 25° C.

Membrane recovery ratio: 75% (four times concentration)

NPOC concentration in concentrated water of test solutions I to III: 29.6 to 30.4 mg/L

The NPOC concentration is a concentration given when the evaluation chemical is not added.

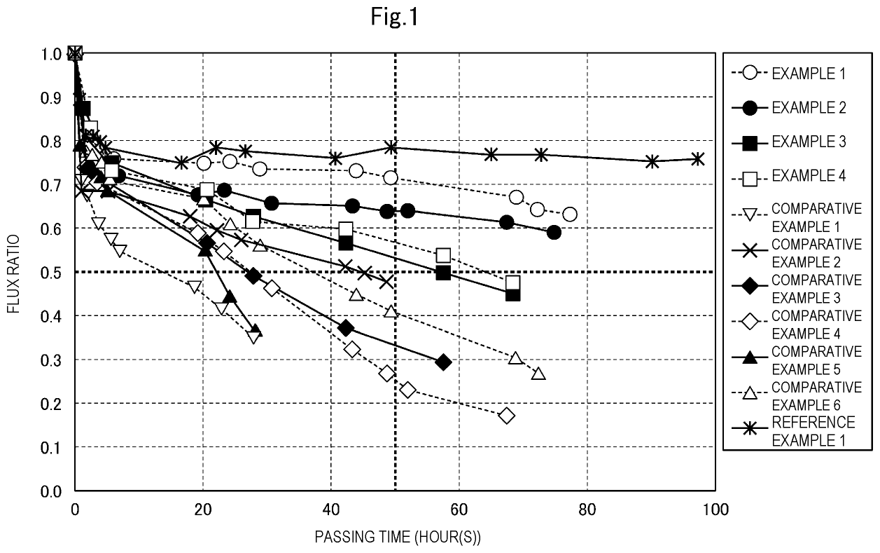

In the above test, changes of a flux ratio (ratio of flux after the elapse of a certain period of time to initial flux) with time were examined, and the results are shown in FIG. 1.

From FIG. 1, the following has become apparent.

In Comparative Example 1 in which the chemical was not added, the flux ratio markedly decreased with time.

Also in Comparative Examples 2 to 6, this decrease was not able to be sufficiently prevented.

In Examples 1 to 4, 50% or more of the initial flux was able to be maintained even in the continuous pass for 50 hours or more.

The chemicals used in Examples 1 to 4 include chemicals publicly known as usual scale dispersants, but the effect of dispersing an organic compound having a phenolic hydroxy group has not been conventionally known.

On the other hand, in the test solution II containing Ca but not containing Canadian Fulvic Acid, a decrease in flux was not observed, and therefore, it can be understood that the effect of the present invention (flux-enhancing effect in comparison with Comparative Example 1 containing no chemical) is an effect of dispersing the organic compound having a phenolic hydroxy group, and is an effect of a different nature from the effect due to the Ca scale preventive agent.

The present invention has been described in detail using a specific embodiment, but it is obvious to a person skilled in the art that various modifications can be made without departing from the spirit and scope of the present invention.

The present application is based on Japanese Patent Application No. 2017-050167 filed on Mar. 15, 2017, and the whole thereof is incorporated herein by reference.

1. A water treatment chemical for a membrane, being a water treatment chemical for preventing membrane contamination caused by an organic compound having a phenolic hydroxy group, and comprising an organic amine having two or more nitrogen atoms and having five or more carboxyl groups or four or more phosphate groups.

2. The water treatment chemical for a membrane according to claim 1, wherein the organic amine is an organic amine represented by any one of the following (1) to (3): 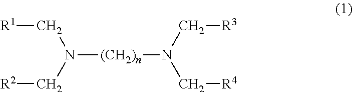
wherein R¹, R², R³ and R⁴ each represent a phosphate group, and n is an integer of 1 or more, 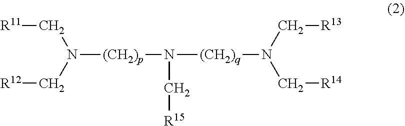
wherein R¹¹, R¹², R¹³, R¹⁴ and R¹⁵ each represent a carboxyl group or a phosphate group, and p and q are each independently an integer of 1 or more, 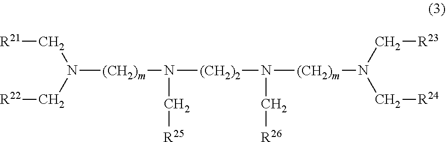
wherein R²¹, R²², R²³, R²⁴, R²⁵ and R²⁶ each represent a carboxyl group, and m is an integer of 1 or more.

3. The water treatment chemical for a membrane according to claim 2, wherein n in the general formula (1), p and q in the general formula (2), and m in the general formula (3) are each an integer of 1 to 5.

4. The water treatment chemical for a membrane according to claim 1, wherein a molecular weight of the organic amine is 100 to 1000.

5. The water treatment chemical for a membrane according to claim 1, being a water treatment chemical for a reverse osmosis membrane for carrying out reverse osmosis membrane treatment of water to be treated, the water containing an organic compound having a phenolic hydroxy group, wherein a concentration of non-purgeable organic carbon in concentrated water obtained by the reverse osmosis membrane treatment is 0.01 to 100 mg/L.

6. The water treatment chemical for a membrane according to claim 5, wherein a concentration ratio of the reverse osmosis membrane is 3 times or more.

7. A membrane treatment method comprising adding the water treatment chemical for a membrane according to claim 1 to water to be treated, the water containing an organic compound having a phenolic hydroxy group, and then subjecting the water to be treated to membrane separation treatment.

8. The membrane treatment method according to claim 7, wherein the water treatment chemical for a membrane is added to the water to be treated in such a manner that the concentration of the organic amine becomes 0.01 to 20 mg/L.
